(19) United States
(12) Patent Application Publication
(10) Pub. No.: US 2024/0222852 A1
(43) Pub. Date: Jul. 4, 2024

(54) RETENTION FEATURES FOR ANTENNA

(71) Applicant: Cardiac Pacemakers, Inc., St. Paul, MN (US)

(72) Inventors: James Michael English, Cahir (IE); Moira B. Sweeney, St. Paul, MN (US); Robert Allen Jones, Lake Elmo, MN (US); David P. Stieper, North Branch, MN (US); Daniela Guerrera, Clonmel (IE); Michael Dunne, Castlecomer (IE); Paul Clarke, Clonmel (IE)

(21) Appl. No.: 18/398,395

(22) Filed: Dec. 28, 2023

Related U.S. Application Data
(60) Provisional application No. 63/435,873, filed on Dec. 29, 2022.

(51) Int. Cl. H01Q 1/27

(52) U.S. Cl. CPC H01Q 1/273; A61N 1/37229

(57) ABSTRACT

An implantable medical device can include a housing including electronic devices within the housing, a header attached to the housing and including one or more bores, an antenna extending from the housing into the header, and an antenna clip configured to position and retain the antenna relative to the header.

### CLAIM OF PRIORITY

This application claims the benefit of U.S. Provisional Application No. 63/435,873 filed on Dec. 29, 2022, which is hereby incorporated by reference in its entirety.

### TECHNICAL FIELD

Various embodiments described herein relate to apparatus, systems, and methods associated with implantable medical devices.

### BACKGROUND

An ambulatory medical device, such as an implantable medical device (IMD), can be configured for implant in a subject, such as a patient. An IMD can be configured to be coupled to a patient's heart such as via one or more implantable leads. Such an IMD can obtain diagnostic information or generate therapy to be provided to the patient, such as via the coupled implantable lead.

In one configuration, IMDs have a header that is coupled to a container that houses much of the electronics of the IMD. The header can be used to couple a conductor of the lead with circuitry within the implantable device. In some examples, an antenna can be placed in the header. However, the antenna requires consistent placement for proper performance.

### SUMMARY

Example 1 can include subject matter such as an implantable device. The implantable device can include a housing including electronic devices within the housing, a header attached to the housing and including one or more bores, an antenna extending from the housing into the header, and an antenna clip configured to position and retain the antenna relative to the header.

In Example 2, the subject matter of Example 1 can optionally include the antenna clip including a channel having an overhang to retain the antenna.

In Example 3, the subject matter of any one or more of Examples 1-2 can optionally include the antenna clip including the overhang on only one side of the channel.

In Example 4, the subject matter of any one or more of Examples 1-3 can optionally include the antenna clip including a cut-out portion in a channel wall opposite the overhang.

In Example 5, the subject matter of any one or more of Examples 1-4 can optionally include the overhang including a protrusion extending from a wall of the channel.

In Example 6, the subject matter of any one or more of Examples 1-5 can optionally include the antenna clip attaching to a post extending from a top of the housing.

In Example 7, the subject matter of any one or more of Examples 1-6 can optionally include the post including a retention head.

In Example 8, the subject matter of any one or more of Examples 1-7 can optionally include the antenna clip including a separate retaining member that is attached the antenna clip so as to overhang a channel in the antenna clip.

In Example 9, the subject matter of any one or more of Examples 1-8 can optionally include the antenna clip including a pair of resilient springs, comprising a first resilient spring extending at least partway over a channel in the antenna clip and a second resilient spring extending from an opposite side of the channel at least partway over the channel, wherein the first resilient spring and the second resilient spring overlap.

In Example 10, the subject matter of any one or more of Examples 1-9 can optionally include the antenna including a single wire extending from a feedthrough assembly.

Example 11 can include an antenna positioning device for an implantable medical device, wherein the antenna positioning device can include an antenna clip attached to a housing of the implantable medical device, a channel extending along the antenna clip for holding an antenna extending from the housing, and a retention member to retain the antenna within the channel relative to a header of the implantable medical device

In Example 12, the subject matter of Example 11 can optionally include the antenna clip including a channel having an overhang positioned to retain the antenna.

In Example 13, the subject matter of any one or more of Examples 1-12 can optionally include the antenna clip including the overhang on only one side of the channel.

In Example 14, the subject matter any one or more of Examples 1-13 can optionally include the antenna clip including a cut-out portion in a wall of the channel opposite the overhang.

In Example 15, the subject matter of any one or more of Examples 1-14 can optionally include the retention member including a separate retaining member that is attached to the antenna clip so as to overhang the channel.

In Example 16, the subject matter of any one or more of Examples 1-15 can optionally include the retention member including a pair of resilient springs, comprising a first resilient spring extending at least partway over the channel and a second resilient spring extending from an opposite side of the channel at least partway over the channel, wherein the first resilient spring and the second resilient spring overlap.

Example 17 can include subject matter such as a method for positioning an antenna within a header of an implantable medical device. The method can include positioning an antenna within a channel of an antenna clip mounted to a housing of the implantable medical device; and physically retaining the antenna within the channel by providing a retention member at least partially over the channel.

In Example 18, the subject matter of any one or more of Examples 1-17 can optionally include the retention member including an overhang located on only one side of the channel, and wherein there is a cut-out portion in a wall of the channel opposite the overhang.

In Example 19, the subject matter of any one or more of Examples 1-18 can optionally include the retention member including a separate retaining member that is attached to the antenna clip so as to overhang the channel.

In Example 20, the subject matter of any one or more of Examples 1-19 can optionally include the retention member including a pair of resilient springs, comprising a first resilient spring extending at least partway over the channel and a second resilient spring extending from an opposite side of the channel at least partway over the channel, wherein the first resilient spring and the second resilient spring overlap.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

FIG. 1 shows an example implantable medical device, in accordance with one embodiment.

FIG. 2 shows a detail of a portion of the implantable medical device, in accordance with one embodiment.

FIG. 3 shows another detail of a portion of the implantable medical device, in accordance with one embodiment.

FIG. 4 shows a view of an antenna clip, in accordance with one embodiment.

FIG. 5 shows a detail of a portion of the antenna clip, in accordance with one embodiment.

FIG. 6 shows a perspective view of the antenna clip. in accordance with one embodiment.

FIG. 7 shows a cross-section view of the channel of an antenna clip, in accordance with one embodiment.

FIG. 8 shows a cross-section view of the channel of an antenna clip, in accordance with one embodiment.

FIG. 9 shows a cross-section view of the channel of an antenna clip, in accordance with one embodiment.

FIG. 10 shows a method of positioning an antenna within a header of an implantable medical device, in accordance with one embodiment.

### DETAILED DESCRIPTION

In the following detailed description, reference is made to the accompanying drawings that form a part hereof and in which are shown, by way of illustration, specific embodiments in which the invention may be practiced. These embodiments are described in sufficient detail to enable those skilled in the art to practice the invention. Other embodiments may be utilized and structural, logical, and electrical changes may be made.

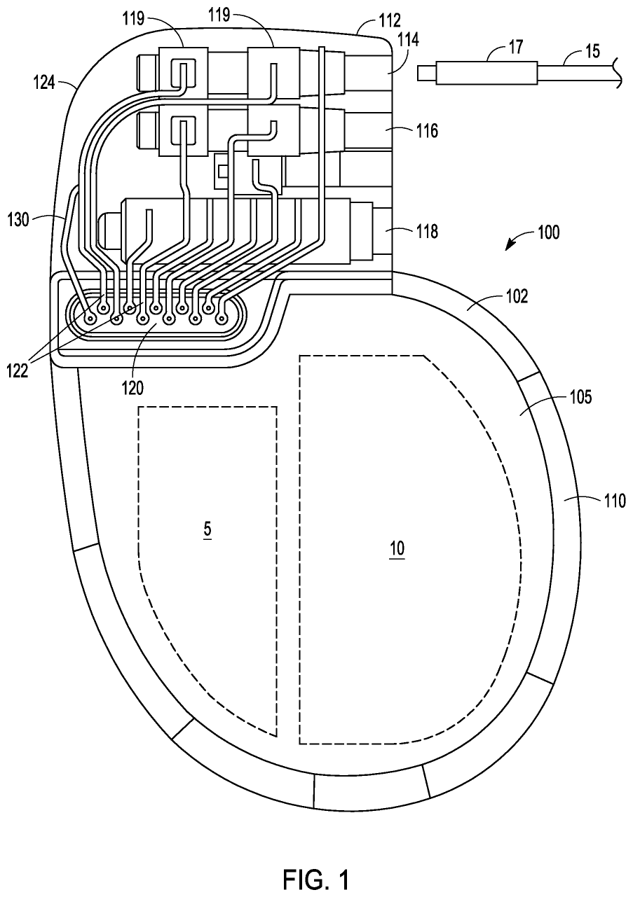

FIG. 1 shows an implantable system 100 including an implantable medical device 102, in accordance with one embodiment. The implantable medical device 102 includes a pulse generator 105 and at least one implantable lead 15. The pulse generator 105 includes a housing 110 and a header 112 mounted to the housing 110. The pulse generator 105 can be implanted into a subcutaneous pocket made in the wall of a patient's chest. Alternatively, the pulse generator 105 can be placed in a subcutaneous pocket made in the abdomen, or in other locations. Pulse generator 105 can include electronic devices such as a power supply 5 including a battery, a capacitor, and other components housed in the housing 110. The pulse generator 105 can further include other electronic devices such as microprocessors 10 to provide processing, evaluation, and to deliver electrical shocks and pulses of different energy levels and timing for defibrillation, cardioversion, and pacing to a heart in response to cardiac arrhythmia including fibrillation, tachycardia, heart failure, and bradycardia.

The header 112 can include one or more bores 114, 116, 118 to receive an implantable lead 15. The implantable lead 15 can include electrodes on a distal end to provide therapy to a body and include a terminal pin 17 on the proximal end to couple to the bore 114, 116, 118. At least one electrical conductor is disposed within the lead 15 and extends from the proximal end to the electrode. The electrical conductor carries electrical currents and signals between the pulse generator 105 and the electrode.

Contacts on the terminal pin 17 can electrically contact electrical contacts 119 within the bores 114, 116, 118 to allow signals and therapy to be delivered to and from electrodes in a body to the electronics 5, 10 within the housing 110. The contacts 119 can be connected by wires 122 to a feedthrough assembly 120 to electrically communicate between the lead 15 and the electronics within the housing 110. The components of the header 112 can be overmolded with a plastic overmold 124 which covers and encapsulates all the components.

In one example, the header 112 can be formed from a polymer material. A polymer can provide a number of desirable features, such as biocompatibility, strength, resilience, and ease of manufacturing. In one example, the header 112 is molded separately from the housing 110, and later bonded to the housing 110 using an adhesive. In a second example, the header 112 can be molded in place (overmolded) and contacts a surface of the housing 110 during a curing or hardening process.

In this example, the implantable medical device 102 includes an antenna 130 extending from the housing 110 into the header 112. The antenna 130 can be coupled on one end to the feedthrough assembly 120 attached to the housing 110. The other end of the antenna 130 extends into the header 112. In this example, the antenna 130 includes a single wire extending from a feedthrough assembly 120.

In other embodiments, the implantable system 100 can also be suitable for use with implantable electrical stimulators, such as, but not limited to, neuro-stimulators, skeletal stimulators, central nervous system stimulators, or stimulators for the treatment of pain.

As noted above, the antenna 130 requires consistent placement within the header 112 for proper performance. Accordingly, the present system provides an antenna clip with antenna wire retention features.

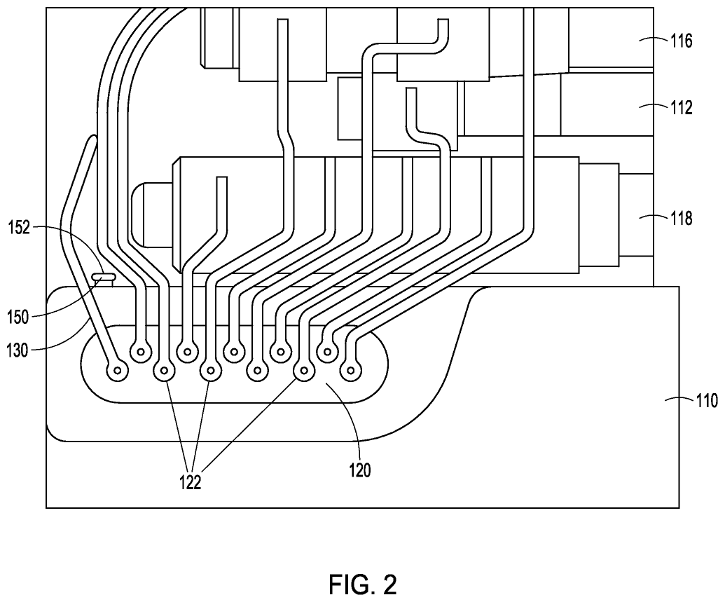

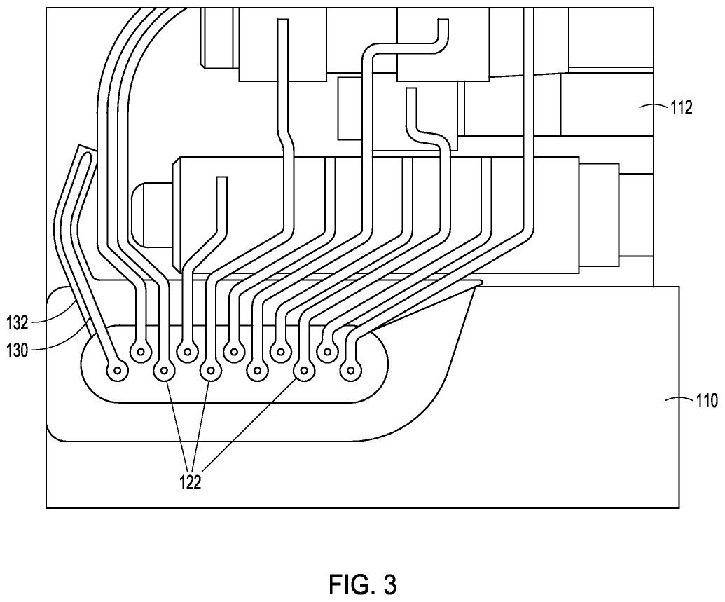

FIG. 2 shows a detail of a portion of the implantable medical device 102, in accordance with one embodiment. In this view, the device is shown without the antenna clip in place, for sake of reference. FIG. 3 shows another detail of the same portion of the implantable medical device, with the antenna clip 132 positioned in the header.

Here, the antenna clip 132 is positioned directly above the housing 110 and is coupled to a top surface of the housing 110 by a post 150. The post 150 can include a retention head 152 which couples to a corresponding slot on the antenna clip 132 to hold the antenna clip 132 in place before the header 112 is overmolded. The antenna clip 132 can be a plastic member shaped and formed to hold the antenna 130 in position before the header 112 is overmolded. In one embodiment, portions of the antenna clip 132 can further include slots to retain sections of the wires 122 also.

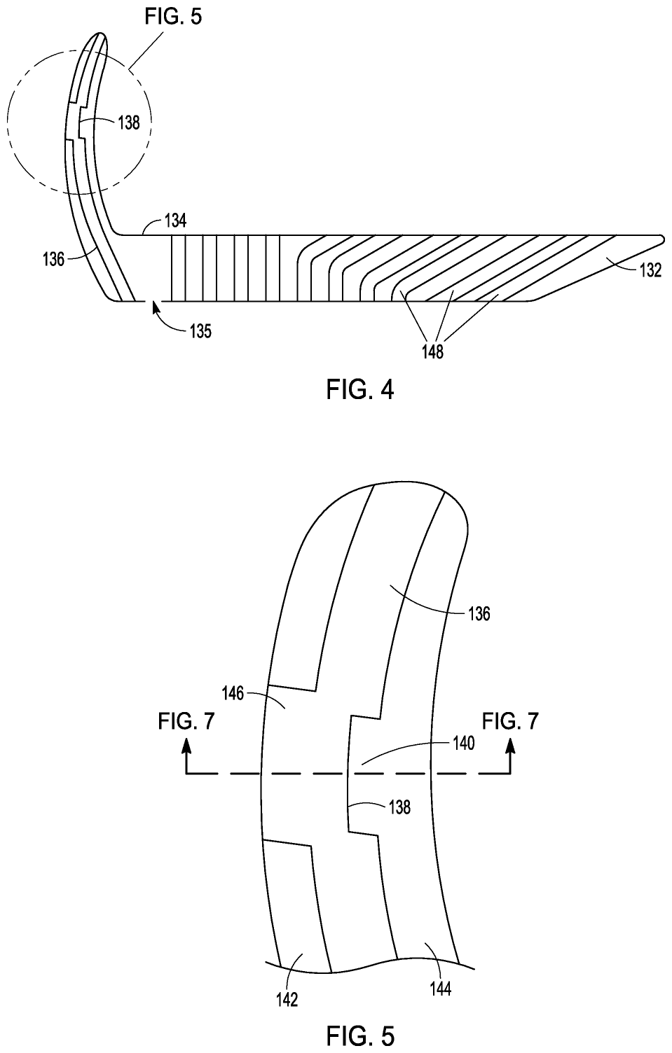

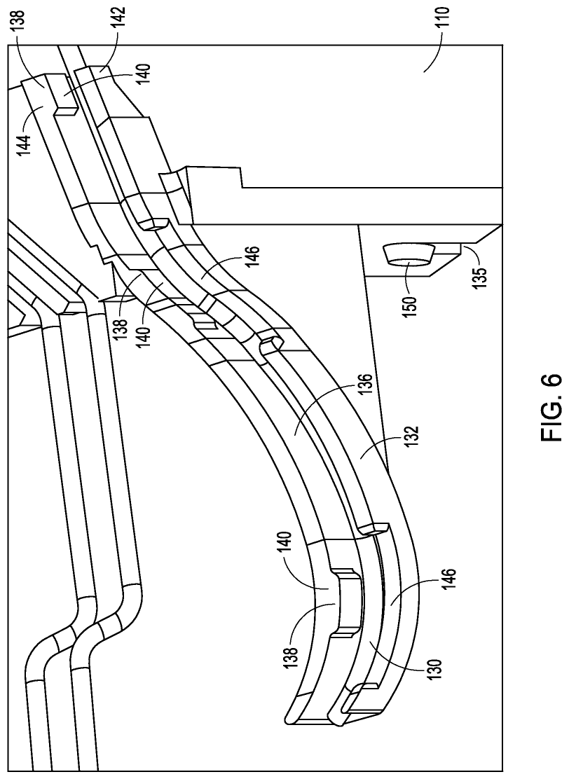

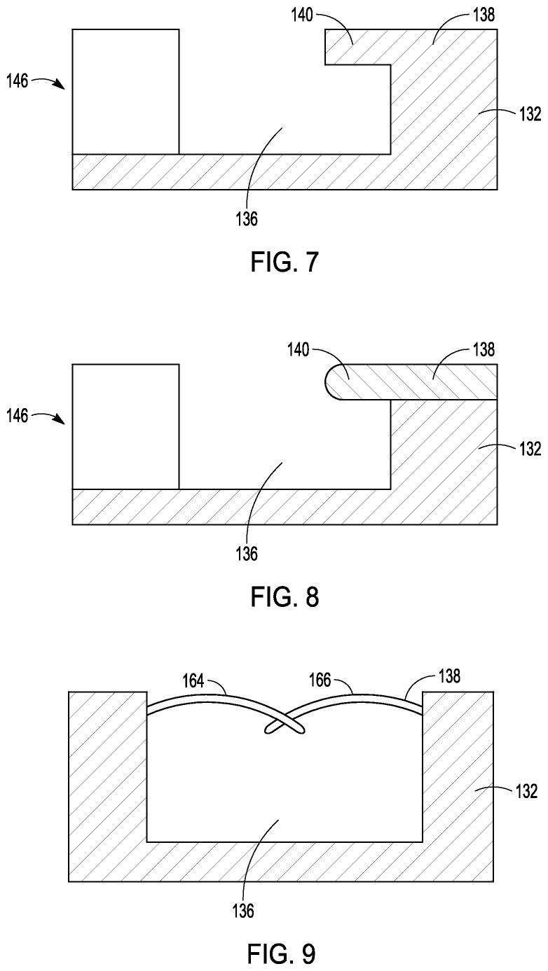

FIG. 4 shows a view of the antenna clip 132, and FIG. 5 shows a detail of a portion of the antenna clip 132, in accordance with one embodiment. FIG. 6 shows a perspective view of the antenna clip 132, and FIG. 7 shows a cross-section view of a channel and the antenna clip 132, in accordance with one embodiment.

Here, the antenna clip 132 can be configured to position and retain the antenna 130 relative to the header. The antenna clip 132 can be mounted to the pulse generator 105 in a variety of manners. For example, as noted above, the antenna clip 132 can include a slot 135 in a body 134 of the antenna clip 132 to couple to the post 150 and retention head 152 of the housing 110. For example, the post 150 can include the retention head 152 and the antenna clip 132 can include the corresponding groove or slot 135 to receive the post 150 and be retained thereby. In other examples, the antenna clip 132 can optionally attach to a post or other feature located on the bore 118 or can be attached to a post or other feature positioned on the feedthrough assembly 120.

In one example, the antenna clip 132 can include a channel 136 having a retention member 138, such as an overhang 140 to physically retain the antenna 130. The channel 136 can be formed in the body 134 of the antenna clip 132 to run elongate up and down the body 134. The channel 136 can include a first wall 142 and a second wall 144. The channel 136 is sized so as to be able to hold the antenna 130 between the first and second walls 142, 144.

In one example, the antenna clip 132 includes the overhang 140 on only one side of the channel 136. In some examples, the overhang 140 can include a protrusion extending from the wall 144 of the channel 136 to at least partially cover over the channel 136. Thus, when the antenna 130 is positioned within the channel 136, the overhang 140 retains the antenna and does not let the antenna 130 slip out of the channel 136 during manufacturing. After all the parts of the header 112 are put together properly, the header 112 can be overmolded to hold out the header components, including antenna 130, in place.

In one example, the antenna clip 132 includes a cut-out portion 146 in the channel wall 142 opposite the overhang 140. The cut-out 146 allows for the user to more easily maneuver the antenna 130 into the channel 136 by providing an outlet for a portion of the antenna 130 as the antenna 130 is being placed within the channel 136 and under the overhang 140. Thus, the cut-out 146 provides a relief section during manipulation of the antenna 130 into the channel 136.

In some examples, as shown in FIG. 6, the antenna clip 132 can include a plurality of retention members 138, such as overhangs 140, along the wall 144. Cutouts 146 can be positioned opposite some of the overhangs 140 while being omitted opposite other overhangs 140.

The antenna clip body 134 can further include a plurality of slots 148 to hold the wires 122 of the device.

FIG. 8 shows a cross-section view of the channel 136 of the antenna clip 132, in accordance with one embodiment. In this example, the antenna clip 132 can include the retention member 138 including a separate retaining member 160 that is attached the antenna clip 132 so as to overhang the channel 136 in the antenna clip 132. The retention member of FIG. 8 works in a similar fashion as the retention member discussed above. In some embodiment, both types of retention members can be utilized.

FIG. 9 shows a cross-section view of the channel 136 of the antenna clip 132, in accordance with one embodiment. In this example the retention member 138 of the antenna clip 132 can include a pair of resilient springs 164, 166, comprising a first resilient spring 164 extending at least partway over the channel 136 in the antenna clip 132 and a second resilient spring 166 extending from an opposite side of the channel 136 at least partway over the channel 136.

The first resilient spring 164 and the second resilient spring 166 can overlap each other. During manufacturing, a user can push the antenna 130 down into the channel 136 through the resilient springs 164, 166. After the antenna 130 has passed the resilient springs 164, 164, each of the springs 164, 166 springs back into place to cover over and retain the antenna 130 within the channel 136 while the rest of the header 112 is manufactured. In this example, the cutout 146 is not provided since the cutout is not needed to maneuver the antenna 130 into the channel 136. Again, in some embodiments, the retention member 138 of FIG. 9 can be combined with the other retention members discussed above.

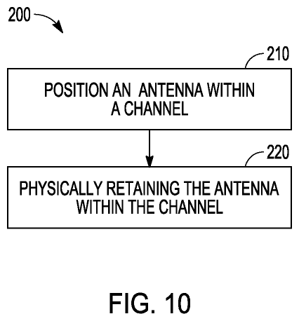

FIG. 10 shows a method (200) of positioning an antenna within a header of an implantable medical device, in accordance with one embodiment. The method (200) can include positioning an antenna within a channel (210) of an antenna clip mounted to a housing of the implantable medical device. The method can further include physical retaining the antenna within the channel (220) by providing a retention member at least partially over the channel.

As discussed above, in various embodiment, the retention member can include an overhang located on only one side of the channel. There can be a cut-out portion in a wall of the channel opposite the overhang. Optionally, the retention member can include a separate retaining member that is attached the antenna clip so as to overhang the channel. Optionally, the retention member can include a pair of resilient springs, comprising a first resilient spring extending at least partway over the channel and a second resilient spring extending from an opposite side of the channel at least partway over the channel, wherein the first resilient spring and the second resilient spring overlap.

As noted, after the other header components are positioned, the entire header can be overmolded to keep all the components in place. Accordingly, the antenna clip of the present disclosure provides for an ease of manufacturing because the user does not have to struggle to keep the wire of the antenna within the channel. Without the retention member to hold the antenna within the channel, it can be easy for the antenna to pop out during placement of other components of the header and before the header is overmolded.

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An implantable medical device comprising:
a housing including electronic devices within the housing;
a header attached to the housing and including one or more bores;
an antenna extending from the housing into the header; and
an antenna clip configured to position and retain the antenna relative to the header.

2. The implantable medical device of claim 1, wherein the antenna clip includes a channel having an overhang to retain the antenna.

3. The implantable medical device of claim 2, wherein the antenna clip includes the overhang on only one side of the channel.

4. The implantable medical device of claim 3, wherein the antenna clip includes a cut-out portion in a channel wall opposite the overhang.

5. The implantable medical device of claim 2, wherein the overhang includes a protrusion extending from a wall of the channel.

6. The implantable medical device of claim 1, wherein the antenna clip attaches to a post extending from a top of the housing.

7. The implantable medical device of claim 6, wherein the post includes a retention head.

8. The implantable medical device of claim 1, wherein the antenna clip includes a separate retaining member that is attached to the antenna clip so as to overhang a channel in the antenna clip.

9. The implantable medical device of claim 1, wherein the antenna clip includes a pair of resilient springs, comprising a first resilient spring extending at least partway over a channel in the antenna clip and a second resilient spring extending from an opposite side of the channel at least partway over the channel, wherein the first resilient spring and the second resilient spring overlap.

10. The implantable medical device of claim 1, wherein the antenna includes a single wire extending from a feedthrough assembly.

11. An antenna positioning device for an implantable medical device, wherein the antenna positioning device comprises:
an antenna clip attached to a housing of the implantable medical device;
a channel extending along the antenna clip for holding an antenna extending from the housing; and
a retention member to retain the antenna within the channel relative to a header of the implantable medical device.

12. The antenna positioning device of claim 11, wherein the antenna clip includes a channel having an overhang positioned to retain the antenna.

13. The antenna positioning device of claim 12, wherein the antenna clip includes the overhang on only one side of the channel.

14. The antenna positioning device of claim 13, wherein the antenna clip includes a cut-out portion in a wall of the channel opposite the overhang.

15. The antenna positioning device of claim 11, wherein the retention member includes a separate retaining member that is attached to the antenna clip so as to overhang the channel.

16. The antenna positioning device of claim 11, wherein the retention member includes a pair of resilient springs, comprising a first resilient spring extending at least partway over the channel and a second resilient spring extending from an opposite side of the channel at least partway over the channel, wherein the first resilient spring and the second resilient spring overlap.

17. A method for positioning an antenna within a header of an implantable medical device, the method comprising:
positioning an antenna within a channel of an antenna clip mounted to a housing of the implantable medical device; and
physically retaining the antenna within the channel by providing a retention member at least partially over the channel.

18. The method of claim 17, wherein the retention member includes an overhang located on only one side of the channel, and wherein there is a cut-out portion in a wall of the channel opposite the overhang.

19. The method of claim 17, wherein the retention member includes a separate retaining member that is attached to the antenna clip so as to overhang the channel.

20. The method of claim 17, wherein the retention member includes a pair of resilient springs, comprising a first resilient spring extending at least partway over the channel and a second resilient spring extending from an opposite side of the channel at least partway over the channel, wherein the first resilient spring and the second resilient spring overlap.
